(19) United States
(12) Patent Application Publication
(10) Pub. No.: US 2024/0033101 A1
(43) Pub. Date: Feb. 1, 2024

(54) FLEXIBLE INTERVERTEBRAL FUSION CAGE WITH OPENING

(71) Applicants: Ming Chi University of Technology, New Taipei City (TW); CHANG GUNG MEMORIAL HOSPITAL, LINKOU, Taoyuan City (TW)

(72) Inventors: PIN-YI CHEN, New Taipei City (TW); PO-LIANG LAI, Taoyuan City (TW); KUEI-CHIH FENG, New Taipei City (TW); CHI-SHUN TU, New Taipei CIty (TW); CHI-YUN WANG, New Taipei City (TW); YU-JIE WU, New Taipei City (TW); SHYANG-YIH KUNG, New Taipei City (TW); GUAN-YI HUNG, New Taipei City (TW)

(21) Appl. No.: 18/103,949

(22) Filed: Jan. 31, 2023

(30) Foreign Application Priority Data
Jul. 27, 2022 (TW) 111128211

(51) Int. Cl. A61F 2/44

(52) U.S. Cl. CPC A61F 2/4455; A61F 2002/3093

(57) ABSTRACT

A flexible intervertebral cage with opening is provided, including a main body, a porous structure part, a side opening, and a rear opening. The porous structure part is arranged at the corresponding positions of the upper and lower parts of the main body; the side opening are located on the two sides of the main body that are different from the upper and lower parts, and the rear opening is located at the other ends of the upper and lower parts of the main body opposite to the end part that connects the upper and lower part. As such, a C-shaped opening structure is used to effectively reduce stress shielding and stress transfer. The biomimetic porous structure of the structural part can promote the attachment and growth of bone cells.

### CROSS-REFERENCE TO RELATED APPLICATION

This application claims the priority of Taiwanese patent application No. 111128211, filed on Jul. 27, 2022, which is incorporated herewith by reference.

### BACKGROUND OF THE INVENTION

### 1. Field of the Invention

The present invention relates generally to an intervertebral fusion cage, and more particularly, to a flexible intervertebral fusion cage with opening.

### 2. The Prior Arts

In modern medicine, putting biomedical implants into living organisms is a fairly mature technology to replace the original joints, intervertebral discs or tooth roots . . . etc. Materials-related research has also become one of the current development priorities in the field of biomedicine.

The vertebral bodies of the vertebrae are not directly and tightly connected, and therefore an intervertebral space exists between the vertebral bodies and an intervertebral disc is arranged in the intervertebral space to support, absorb, and slow down the pressure or shock on the vertebral bodies. For some spine-related conditions, such as spondylolisthesis, herniated intervertebral disc (HIVD), or spinal stenosis that compresses nerves, it is usually necessary to remove the entire or part of the intervertebral disc by surgery, and then a structure that can support the upper and lower vertebral bodies is implanted into the intervertebral space after the intervertebral disc resection to avoid the collapse of the intervertebral space after the intervertebral discectomy or resulting in spinal deformation and other adverse consequences. It can be seen that in order to restore the intervertebral space to maintain the height and to push the vertebral body back to a near-normal position, the implant placed in the intervertebral space needs to be properly selected.

The existing fusion cage needs to overcome the stress shielding effect caused by the excessive elastic modulus of the titanium alloy, and most of the existing fusion cages do not have biological activity and cannot form a good biological link.

As such, the inventor came to the present invention after observing the aforementioned issues.

### SUMMARY OF THE INVENTION

In order to achieve the above objective, the present invention provides a flexible intervertebral fusion cage with opening, which is applicable to chiropractic treatment and assists bone hyperplasia. The flexible intervertebral fusion cage with opening mainly comprises a main body, a porous structure part, a side opening and a rear opening.

The side opening and the rear opening of the flexible intervertebral fusion cage with opening roughly divide the main body into an upper part and a lower part, with junction thereof as an end part.

The porous structure part is disposed at corresponding positions of the upper and lower parts of the main body, and more precisely, is embedded in the corresponding positions of the upper and lower parts of the main body.

The side opening is formed by the upper part, lower part, and the end part of the main body, and the rear opening is located at the other end of the upper and lower parts of the main body opposite to the end part; that is, one end of the upper and lower parts of the main body is formed by the end part and the other ends not connected to each other form the rear opening.

The porous structure part is composed of biological scaffolds with a porous structure, and the diameter of the pores of the porous structure part is preferably 0.8-1.2 mm.

Preferably, the end of the main body is wedge-shaped.

In order to make those skilled in the art understand the purpose, features and effects of the present invention, the present invention is described in detail as follows by means of the following specific embodiments and in conjunction with the accompanying drawings.

### BRIEF DESCRIPTION OF THE DRAWINGS

FIG. 1 is a schematic view of a flexible intervertebral fusion cage with opening according to the present invention;

FIG. 2 is a schematic side view of the flexible intervertebral fusion cage with opening according to the present invention;

FIG. 3 is a schematic view of the porous structure part of the flexible intervertebral fusion cage with opening according to the present invention;

FIG. 4A is a schematic diagram of stress analysis of the prior art;

FIG. 4B is a schematic diagram of stress analysis of a flexible intervertebral fusion cage with opening according to the present invention;

FIG. 5A is another schematic diagram of stress analysis of the flexible intervertebral fusion cage with opening according to the present invention;

FIG. 5B is another schematic diagram of stress analysis of the flexible intervertebral fusion cage with opening according to the present invention; and

FIG. 6 is another schematic view of the flexible intervertebral fusion cage with opening according to the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

The terminology used herein is used to describe particular embodiments only, and is not intended to limit the present invention. As used herein, the singular terms “a” and “the” are intended to include the plural forms as well, unless the context clearly dictates otherwise. As used herein, the term “and/or” includes any and all combinations of one or more of the associated listed items. It will be understood that when an element is referred to as being “connected” or “coupled” to another element, it can be directly connected or coupled to the other element or intervening elements may be present.

Exemplary embodiments are described herein with reference to the accompanying drawings, which are schematic illustrations of idealized examples. Accordingly, deviations from the shapes shown, for example, caused by manufacturing techniques and/or tolerances, are expected. Thus, the regions illustrated in the figures are schematic and their shapes are not intended to illustrate the actual shape of a region of a device and are not intended to limit the scope of exemplary embodiments.

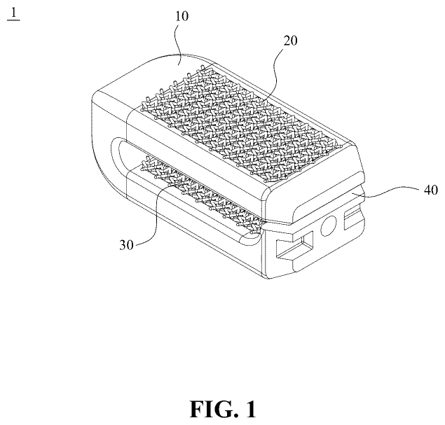

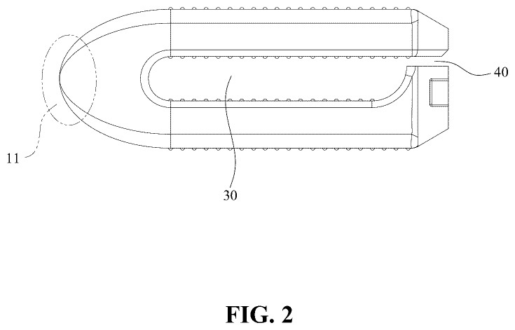

Refer to FIG. 1 and FIG. 2. FIG. 1, which are a schematic view and a side view of the flexible intervertebral fusion cage with opening according to the present invention. The flexible intervertebral fusion cage 1 mainly includes a main body 10, a porous structure part 20, a side opening 30, and a rear opening 40, wherein the main body 10 is divided into an upper part and a lower part, and one end of the upper part and one end of the lower part are connected, referred to as end part 11. The porous structure part 20 is disposed at the corresponding positions of the upper and lower parts of the main body 10, and more precisely, embedded in the corresponding positions of the upper and lower parts of the main body 10.

The side opening 30 is enclosed and formed by the upper and lower parts of the main body 10 and the end part 11. The rear opening 40 is located at the other end of the upper and lower parts of the main body 10 opposite to the end part 11; that is, one end of each of the upper and lower parts of the main body 10 is connected to from the end part 11, and the other end not connected to each other forms a rear opening 40.

Specifically, the appearance of the flexible intervertebral fusion cage 1 can be further referred to in FIG. 2, which is a schematic side view of the flexible intervertebral fusion cage with opening according to the present invention. The side opening 30 and the rear opening 40 of the flexible intervertebral fusion cage 1 roughly divide the main body 10 into the upper and lower parts, and the end part 11 connects the upper and lower parts of the main body 10; alternatively, it can be viewed as the flexible intervertebral fusion cage 1 forming a C-shaped structure when viewed from the side. The design of the opening enables the flexible intervertebral fusion cage 1 to effectively reduce stress shielding and stress transfer.

Specifically, the size of the rear opening 40 is approximately between 0.5-4 mm; that is, the distance between the other ends of the upper part and the lower part is between 0.5-4 mm.

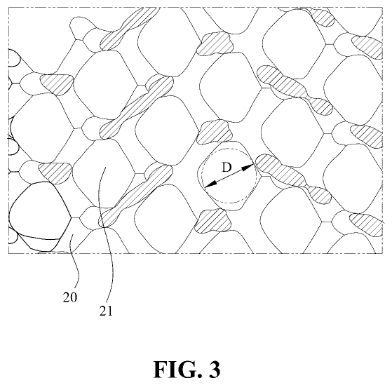

Specifically, refer to FIG. 3, which is a schematic view of the porous structure part of the flexible intervertebral fusion cage according to the present invention. The porous structure part 20 is mainly composed of a biological scaffold with a porous structure. As shown in FIG. 3, the diameter D of holes 21 of the porous structure part 20 is preferably 0.8-1.2 mm.

Specifically, the biomimetic porous structure of the porous structure part 20 can promote the attachment and growth of bone cells.

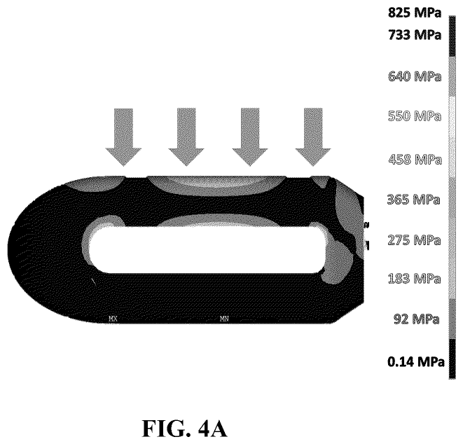

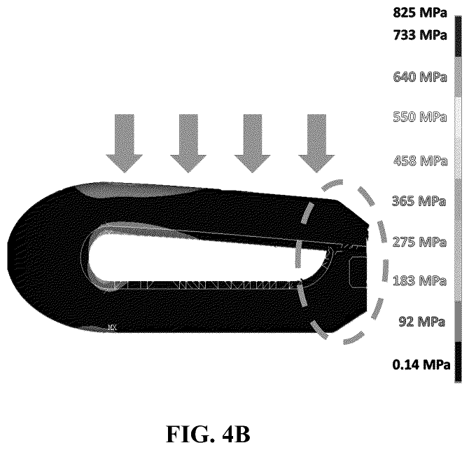

According to the flexible intervertebral fusion cage 1 of the present invention, an actual comparison experiment is performed, as shown in FIGS. 4A and 4B. It can be clearly seen that the side opening 30 and the rear opening 40 are important feature to the present invention.

FIG. 4A is a stress analysis diagram of a cage without the rear opening 40, and it shows the contact surface has a higher stress and is easy to form stress concentration. FIG. 4B is a stress analysis of a flexible intervertebral fusion cage 1 with an opening of the present invention. With the design of the rear opening 40, the stress of the contact surface can be effectively transferred, and at the same time, the flexible intervertebral fusion cage 1 with an opening is deformed by force to produce a high anterior high and a low posterior, which conforms to the human lumbar lordosis structure. As such, the flexible intervertebral fusion cage 1 is more stable in the human body.

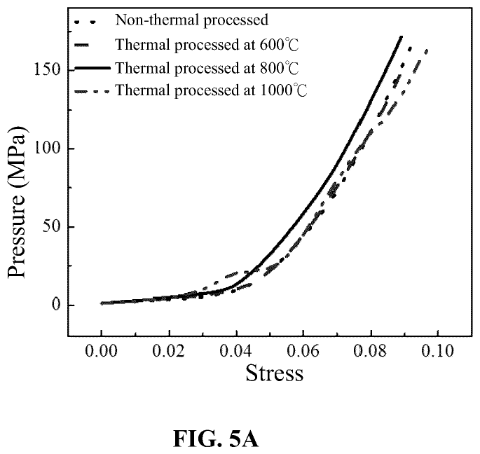

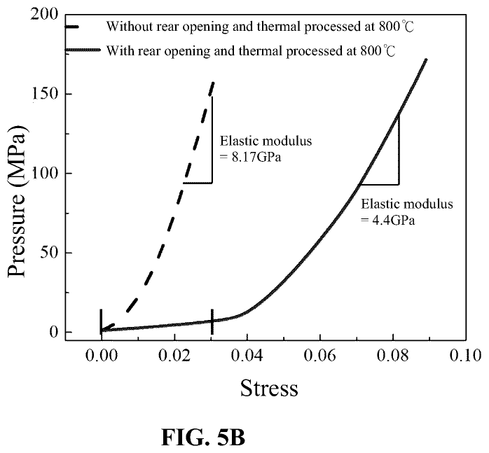

FIGS. 5A and 5B are another stress test and analysis schematic diagrams of the flexible intervertebral fusion cage with opening of the present invention. Through the mechanical strength testing machine to the flexible intervertebral fusion cage with opening, the actual stress test is performed on the flexible intervertebral fusion cage 1, and the test lasts until the flexible intervertebral fusion cage 1 is damaged or the load (N, Newton) reaches the ASTM standard value to terminate the test.

FIG. 5A is a comparison of the compressive strength tests of the flexible intervertebral fusion cage 1 of the present invention under different heat treatment temperatures, wherein the flexible intervertebral fusion cage 1 subjected to different heat treatment temperatures, for example, removing residual stress and changing phase microstructure, has better mechanical strength properties at 800±50° C. due to the microstructure at 800±50° C. α+β phase transforms into a layered structure. This layered structure helps to improve mechanical toughness, while the R phase transforms into needle-like and coarse structures under heat treatment at 600° C. and 1000° C., respectively, resulting in poor overall mechanical properties. FIG. 5B is a comparison diagram of the flexible intervertebral fusion cage with the rear opening 40 and without the rear opening 40. It can be found that the intervertebral fusion cage with the rear opening 40 has a lower elastic modulus, which can effectively reduce the stress shielding effect and has a larger amount of strain to provide the function of human body shock absorption.

Further, the end part 11 of the main body 10 is designed as solid wedge-shaped so that the flexible intervertebral fusion cage 1 can be easily implanted, and the porous structure part 20 is composed of a bio-scaffold with a porous structure. It can increase the surface area to promote cell adhesion, proliferation and allow the circulation of tissue fluids.

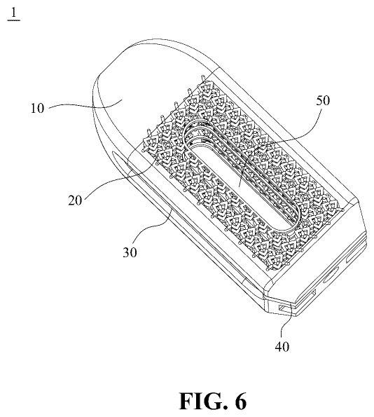

Refer to FIG. 6 for another embodiment of the present invention. The flexible intervertebral fusion cage 1 includes a main body 10, a porous structure part 20, a side opening 30, a rear opening 40, and two through holes 50, with each disposed in the center of the porous structure part 20, and the positions of the through holes 50 of the upper and lower porous structure parts 20 correspond to each other, and the through holes 50 increase more surface area and space to promote bone growth.

In summary, the technical features of the present invention and the technical effects that can be achieved are as follows:

First, with the flexible intervertebral fusion cage with opening of the present invention, the wedge-shaped design of the end part enables the flexible intervertebral fusion cage with opening to be easily implanted.

Second, according to the flexible intervertebral fusion cage with opening of the present invention, the porous structure part increases the surface area to promote cell adhesion, proliferation and allow interstitial fluid to flow.

Third, according to the flexible intervertebral fusion cage with opening of the present invention, after the opening is designed, the stress of the stress shielding and transferring the autologous bone contact surface is effectively reduced, and the cage is deformed by force to produce a high front and a low back. It conforms to the structure of human lumbar lordosis, which can stabilize the implant in the body. In addition, the design of the porous structure can promote the attachment and growth of bone cells.

The above is to illustrate the implementation of the present invention through specific embodiments, those skilled in the art can easily understand other advantages and effects of the present invention from the content disclosed in this specification.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A flexible intervertebral fusion cage with opening, comprising:
a main body, divided into an upper part and a lower part, and the upper part and a lower part being connected by an end part;
a porous structure part, disposed at corresponding positions of the upper and lower parts of the main body;
a side opening, formed by the upper part, lower part and the end part of the main body; and
a rear opening, located at the other end of the upper part and lower part opposite to the end part;
wherein one end of the upper part and one end of the lower part being connected by the end part and the other ends of the upper and lower part not connected to each other forming the rear opening.

2. The flexible intervertebral fusion cage with opening according to claim 1, wherein the porous structure part is composed of bio-scaffolds with a porous structure.

3. The flexible intervertebral fusion cage with opening according to claim 2, wherein the diameter of the pores of the porous structure part is between 0.5 and 1.2 mm.

4. The flexible intervertebral fusion cage with opening according to claim 1, wherein the size of the rear opening is between 0.5-4 mm.
